(19) United States
(12) Patent Application Publication
(10) Pub. No.: US 2006/0216338 A1
(43) Pub. Date: Sep. 28, 2006

(54) Composition and method of treatment for heel pain

(76) Inventor: W. J. Easterling, San Antonio, TX (US)

Correspondence Address: NAMAN, HOWELL, SMITH & LEE, P.O. BOX 1470, WACO, TX, 76703, US

(21) Appl. No.: 11/320,508

(22) Filed: Dec. 28, 2005

Related U.S. Application Data
(60) Provisional application No. 60/650,833, filed on Feb. 7, 2005.

(51) Int. Cl. A61K 9/70; A61K 31/554; A61K 31/455

(52) U.S. Cl. 424/449; 514/355; 514/211.07

(57) ABSTRACT

A topical medicament and associated methodology for use thereof, through the use of which tissue disorders involving collagen degeneration associated with repetitive microtears of the plantar fascia may be effectively, cost effectively, and painlessly treated. One or more calcium channel blocker agents serve as the primary active ingredient of the present compositions, with carrier agents facilitating non-invasive transdermal delivery of the calcium channel blocker(s) to sub-dermal disease sites.

### CITATION TO PRIOR APPLICATION

This application claims the benefit of U.S. Provisional Application No. 60/650,833, filed on Feb. 7, 2005.

### BACKGROUND OF THE INVENTION

1. Field of the Invention

The present invention generally relates to a composition and method of treatment for heel pain. More specifically, the present invention relates to a composition and method of treatment for heal pain involving the topical application of a Verapamil-containing solution.

2. Background Information

The treatment of heel pain is known in the art. More specifically, heel pain treatment regimens are known to consist basically of familiar, expected and obvious structural configurations, notwithstanding the myriad of designs encompassed by the prior art which have been developed for the fulfillment of different objectives and requirements. While these regimens may fulfill their respective, particularly claimed objectives and requirements, the aforementioned regimens do not disclose a composition and method of treatment for heel pain such as Applicant's present invention.

The plantar fascia is a thickened fibrous aponeurosis that originates from the medial tubercle of the calcaneus and runs forward to form the longitudinal foot arch. The function of the plantar fascia is to provide static support of the longitudinal arch and dynamic shock absorption. Individuals with pes planus (low arches or flat feet) or pes cavus (high arches) are at increased risk for developing plantar fasciitis, a common cause of heel pain in adults. Plantar Fasciitis typically causes pain that is particularly severe with the first few steps taken in the morning. In general, plantar fasciitis is a self-limited condition. However, symptoms usually resolve more quickly when the interval between the onset of symptoms and the onset of treatment is shorter.

The pain associated with Plantar fasciitis is usually caused by collagen degeneration associated with repetitive microtears of the plantar fascia (which is sometimes misnamed chronic inflammation) at the origin of the plantar fascia at the medial tubercle of the calcaneus; this pain is exacerbated by passive dorsiflexion of the toes or by having the patient stand on the tips of the toes. This degeneration, which features loss of collagen continuity, is similar to the chronic necrosis of tendonosis and increases in ground substance (matrix of connective tissue) and vascularity, and the presence of fibroblasts rather than the inflammatory cells usually seen with the acute inflammation of tendonitis. The cause of the degeneration is repetitive microtears of the plantar fascia that overcome the body's ability to repair itself.

As mentioned, the classic sign of plantar fasciitis is that the worst pain occurs with the first few steps in the morning, but not every patient will have this symptom. Patients often notice pain at the beginning of activity that lessens or resolves as they warm up. The pain may also occur with prolonged standing and is sometimes accompanied by stiffness. In more severe cases, the pain will also worsen toward the end of the day.

Anatomic risks associated with Plantar fasciitis include overpronation, discrepancy in leg length, excessive lateral tibial torsion, and excessive femoral anteversion. Functional risk factors include tightness and weakness in the gastrocnemius, soleus, Achilles tendon, and intrinsic foot muscles.

Overuse, rather than anatomy, is the most common cause of plantar fasciitis in athletes. A history of an increase in weight-bearing activities is common, especially those involving running, which causes microtrauma to the plantar fascia and exceeds the body's capacity to recover. Plantar fasciitis also occurs in elderly adults. In these patients, the problem is usually more biomechanical, often related to poor intrinsic muscle strength and poor force attenuation secondary to acquired flat feet and compounded by a decrease in the body's healing capacity.

Diagnostic testing is rarely conducted for the initial evaluation and treatment of plantar fasciitis. Plantar fasciitis is often called heel spurs, although this terminology is somewhat of a misnomer because 15 to 25 percent of the general population without symptoms have heel spurs and many symptomatic individuals do not. Heel spurs are bony osteophytes that can be visualized on the anterior calcaneus on radiography. However, diagnostic testing is indicated in cases of atypical plantar fasciitis, in patients with heel pain that is suspicious for other causes.

The most commonly accepted treatment simply involves rest. Nevertheless, such a regimen remains far from satisfactory. Athletes, active adults, and persons whose occupations require lots of walking are unlikely to comply with such a regimen. Moreover, known treatment regimens have failed in the face of problems that place individuals at risk for plantar fasciitis, such as increased amount of weight-bearing activity, increased intensity of activity, hard walking/running surfaces, and the type of shoes worn.

Other known treatment regimens are based on stretching, strengthening, change of shoes, arch supports, orthotics, night splints, anti-inflammatory agents, and surgery. These regimens, however, are limited in so much as their associated success rate is far from constant. For instance, elderly patients may simply be unable to participate in strengthening or stretching programs as a result of physical limitations. Further, the use of appropriate arch supports and similar products can often be confusing for the uninformed consumer. Such products are highly variable with regard to function and effectiveness. Heel cups, for example, have been found by those skilled in the art to be particularly ineffective in the treatment of plantar fasciitis. Effective orthotics involve a lengthy development process and are typically cost prohibitive. The use of night splints is widely considered disadvantageous as they all too often interfere with the patient's or a bed partner's ability to sleep. Anti-inflammatory drugs in chronic inflammatory diseases is somewhat controversial, and related health risks are many, including gastrointestinal bleeding, gastric pain and renal damage. Iontophoresis, the use of electric impulses from a low-voltage galvanic current stimulation unit to drive topical corticosteroids into soft tissue structure, to be effective, must be administered by an athletic trainer or physical therapist at least two to three times per week. Thus, iontophoresis use is probably best reserved for the treatment of elite athletes and of laborers with acute plantar fasciitis whose symptoms are preventing them from working. Finally, in cases that do not respond to any conservative treatment, surgical release of the plantar fascia may be considered. Risk factors include flattening of the longitudinal arch and heel hypoesthesia as well as the potential complications associated with rupture of the plantar fascia and complications related to anesthesia.

### SUMMARY OF THE INVENTION

The general purpose of the present invention, which will be described subsequently in greater detail, is to provide a composition and method of treatment for heel pain which has many of the advantages of such methods known in the art and many novel features that result in a composition and method of treatment which is not anticipated, rendered obvious, suggested, or even implied by any of the known compositions or methods of treatment, either alone or in any combination thereof. In accomplishing such, the present invention avoids the most common pitfalls associated with know treatment regimens. That is, the present regimen may be administered independently by the patient, is painless, has an extremely low associated risk, is independently effective, and is cost effective.

In satisfaction of the above, the present invention provides a composition and method of treatment for heel pain. The present invention, by way of a novel composition and a particularly effective method of applying that composition, yields results that simply are not possible with any other known treatments. Moreover, the composition and method of treatment, while producing outstanding results when used independently, may be effectively combined with other known treatments. In such cases, results associated with such known treatment dramatically increase.

The invention, although exemplified by specific embodiments which are based upon, or rely on specific calcium channel blockers, is not limited to such species. Therefore, the true scope of the invention encompasses preparations and methods of use involving transdermal application of calcium channel blockers in the treatment of tissue disorders which exhibit dermal and sub-dermal disease.

The medicament of the present invention is a topical gel which has repeatedly effected, in many cases, a complete reversal of perceptible plantar fasciitis symptoms and a substantial reduction of such symptoms to a substantially greater degree and substantially higher incidence than previously experienced by patients. All observations of efficacy of the present compositions and methods arise from physician-supervised and prescribed treatment regimens involving use of the medicament of the present invention. In most cases, use of the present medicaments and prescribed treatment regimens follow prior, wholly or substantially unsuccessful attempts to treat patients' plantar fasciitis. Recent studies involve the treatment of heel pain, most commonly resulting from plantar fasciitis, through use of the medicaments taught herein. These studies strongly suggest the use of topical calcium channel blocker medicaments taught herein are highly effective in treating heel pain appearing to relate to, or being caused by, collagen degeneration associated with repetitive microtears of the plantar fascia.

The preferred form of the present invention involves application of between 0.5-1.0 mL of Verapamil (15% solution) between one and two times per day. Length of treatment varies from one to several months, with a means treatment period being 3 months. Patients are either examined or interviewed and counseled at least every two weeks in order to evaluate progress and monitor side effects. To date, there have been no reported side effects.

### BRIEF DESCRIPTION OF THE DRAWINGS

N/A

### DETAILED DESCRIPTION OF THE PREFERRED EMBODIMENT

A. Preparation of Calcium Channel Blocker Preparations

In the preferred embodiment, the primary active ingredient of the topical gel is a diphenylamine. However, it should be understood that other calcium channel blockers (topically applied in a similar composition) provide similar relief. The presently preferred embodiment of the present medicament utilizes Verapamil Hydrochloride, USP calcium channel blocker of choice. With certain patients, combinations of channel blocker agents seem to have an even greater efficacy than a single such agent. An example of such a combination includes a mixture of roughly equal proportions of a diphenylalkylamine (Verapamil Hydrochloride, USP, for example) and a dihydropyridines (Nifedipine, for example).

Other calcium channel blockers include benzothiazepines (Diltiazem, for example), other dihydropyridines (Amlodipine, Felodipine, Isradipine, Nicardipine, Nimodipine, or Nisoldpine), and the fast sodium inward channel inhibitor—Bepridil.

The preferred Verapamil-based gel of the present invention includes two constituent preparations: Lecithin Isopropyl Myristate Reagent and Pluronic F1 27 Gel 20%. Preparation of the presently preferred embodiment and the best mode of the composition proceeds as follows (with alterations needed for larger scale production, such as by pharmaceutical companies, being within the scope of the invention):

Ingredients:

Verapamil HCL, USP, NDC, 51552-525-50 CAS 152-114

Ethoxy Diglycol Reagent CAS 111-90-0

Poloxamer, NF (407) (Pluronic F127) NDC 38779-0834-8 CAS 9003-11-6

Lecithin, NF 18 (Granular) NDC 38779-0947-3 CAS 8002-43-5

Isopropyl Myristate, NF CAS 110-27-0

Sterile Water for Irrigation NDC 0074-7139-09

Potassium Sorbate, NF NDC 38779-0833-5 CAS 590-00-1

Sorbic Acid, NF-FCC Powder CAS 110-44-1
Note: NDC and CAS identification numbers vary with manufacturers/suppliers. The numbers shown above are representative examples only.
   Preparation of Lecithin Isopropyl Myristate Reagent, 1,100 mL:
1. Lecithin, NF 500 Gm
2. Isopropyl Myristate, NF (Cosmetic Grade) 532 mL
3. Sorbic Acid, NF-FCC Powder 2.7 Gm

Place Lecithin and Sorbic Acid in a glass beaker sufficient to hold 1,100 ml of liquid. Pour Isopropyl Myristate over Lecithin and Sorbic Acid to disperse therein. Cover and allow to sit at room temperature until a smooth syrupy liquid is formed. Stir well and transfer to an amber, light-resistant glass container.

Prepare Poloxamer (407) (Pluronic F127) Reagent, 20%, 800 mL:

1. Poloxamer (407) (Pluronic F127) 160 Gm
2. Potassium Sorbate, NF 2.4 Gm
3. Sterile Water for Irrigation qs 800 mL

Place the Poloxamer (407) (Pluronic F127) and the Potassium Sorbate in a 1000 mL glass beaker. Pour the Sterile Water for Irrigation over these reagents sufficient to show 800 mL on the beaker container. Stir sufficiently to wet the dry reagents with the water. Place in a scientific grade, digital temperature-controlled refrigerator atmospherically controlled between 39 and 42 degrees Centigrade. Allow to stand until all reagents have dissolved in the water. Add additional sterile water for irrigation sufficient to yield 800 ml. Stir well and transfer to a clean glass or plastic container. This reagent must be stored in the refrigerator at the above designated temperature range.

Preparation of Topical Verapamil 80 mg/mL Final Product:

1. Weigh 4.8 GM Verapamil HCl, USP and place in a 100 mL glass beaker.
2. Add 7 mL Ethoxy Diglycol Reagent to Verapamil and stir well.
3. Place Verapamil/Ethoxy Diglycol Reagent mixture on a laboratory grade hot plate that has been pre-heated between 60-80 degrees Centigrade. Stir periodically until all Verapamil is dissolved and a clear solution exists.
4. Remove Verapamil solution from hot plate and add 16 mL of the pre-prepared Lecithin Isopropyl Myristate reagent and stir well. Using a 16G needle luer-locked to a 60 cc syringe, transfer the Verapamil/Lecithin Isopropyl Myristate suspension from the beaker to the 60 cc syringe.
5. Remove the Poloxamer (407) (Pluronic F127) Reagent, 20% from the refrigerator and draw up 34 mL into a second 60 cc luer-lock syringe.
6. Using a Luer Lock To Luer Lock Adapter (Baxa 13901), attach the two 60 cc syringes containing the reagents (one syringe to each side of the Adapter) in preparation for mixing the two together.
7. With adequate pressure applied to the plunger of each 60 cc syringe, force the materials from one syringe to the other, back and forth, at least 25 times, until a smooth, consistent, and creamy mixture is prepared. Discard the empty 60 cc syringe. The final mixture should be pH 5.8-6.2 (pH 6.0 is ideal). Phosphate buffer solution may be used to adjust the pH of this product.
8. Remove the Luer Lock To Luer Lock Adapter from the 60 cc syringe containing the Verapamil mixture and cap the syringe. Protect from light by placing in an amber zip-lock bag for storage.
9. Dispense in 0.5 or 1.0 mL amber-colored plastic containers fitted with an adequate cap for patient use. Place containers in a brown zip-lock bag for further light protection. An example is a 1.0 mL amber syringe (Baxa Oral-Topical Exacta-Med Dispenser) with blue self-uprighting plastic cap. A Baxa Luer Lock-to-Oral Slip #42703 Adapter may be used to transfer the Verapamil compound from the 60 cc syringe to the 1.0 cc Baxa Exacta-Med Dispensers.
10. Label (or, as appropriate, provide patient information sheets containing the following information)
   Topical Administration of Verapamil 40 mg/0.5 ml for Plantar Fasciitis:
      Do Not Refrigerate
      Protect From Light
      For External Use Only
      This medication must not be refrigerated. Refrigeration may destroy the absorption qualities of the carrier agent(s).
      Important: Do not take supplemental Vitamin C while using this medication. It may counteract the results.
      Avoid tea and purple-skinned fruit since they may contain chemicals called anthocyanins that interfere with the desired action of the drug.
      Take at least 500 mg Calcium twice a day with food and an 8 oz glass of water. Calcium citrate may settle better on the stomach.
      Take 50 mg Zinc once a day with food.

Preparation of other calcium channel blocker-based topical medicaments useful in treating similar disorders is not substantially different (if at all), from the preparation of the above-described Verapamil-based gel. Step-by-step procedures for every variation and combination of calcium channel blockers in a topical medicament which itself, and its method of use, would be within the scope of the present invention is both unnecessary to provide an enabling disclosure and is unnecessary from a practical standpoint.

The presently preferred embodiment of a calcium channel blocker-based medicament according to the present invention is as described above with respect to Verapamil. Furthermore, any compounding pharmacist of typical skill can, once exposed to the information taught herein, readily prepare calcium channel blocker-based medicaments for use in practicing the present invention, regardless of the specific identities of the prescribed calcium channel blocker(s). Nevertheless, a benzothiazepine-based medicament, using Diltiazem as the active ingredient, is prepared described below.

Preparation of Diltiazem 100 mg/ml Topical Yield is 60 mL:

1. Weigh 6.0 grams of Diltiazem powder in a glass beaker. Add 5.0 mL of sterile water for irrigation and stir well. Place on a laboratory hot plate preheated between 50-70 degrees centigrade and stir until Diltiazem is dissolved and a clear solution exists.
2. Add 2.0 mL propylene glycol and stir well.
3. Add 2.5 ml 5-Ethyl-2,8-diisopropyl-1-aza-3,7-dioxabicyclo[3.3.0]octane and stir well.
4. Draw solution into a 60 mL sterile syringe using a 16G-1″ needle. Remove needle.
5. Draw 16 mL Lecithin Isopropyl Myristate Reagent into a 30 mL sterile syringe and add to the 60 mL syringe containing the Diltiazem solution using a 16G-1″ needle to make the transfer.
6. Mix the ingredients in the 60 mL syringe well by gentle inversion.
7. Draw 30 mL of 20% Pluronic F-127 Organogel into a second 60 mL sterile syringe.
8. Using a luer lock to luer lock adapter, connect the two 60 mL syringes containing reagents.
9. By pushing the ingredients back and forth between the two 60 mL syringes, using force, perform this operation until a clear consistent gel exists. This usually requires 50-60 transfers from one syringe to the other.
10. Dispense in 3 mL amber oral syringes and store at room temperature.
   B. Use of Medicaments in Treating Plantar Fasciitis

Calcium channel blocker medicaments prepared to be used according to the present invention are, according to the preferred mode of use, dispensed in one-ml amber syringes that are graduated in 0.01 ml increments with major graduations at 0.1 through 1.0 ml. Each syringe is filled to the 1.0 ml mark. One dose (40 mg) of Verapamil-based gel is contained in 0.5 ml. Each syringe is capped with a tip that can be removed and replaced by simply pushing and pulling with a twist.

The patient is to apply 0.5 ml (40 mg) twice a day, in the morning and after a shower in the evening. The old dose must be completely removed and the area cleaned and dried before a new dose is applied. One syringe will last one (1) day.

The patient removes the cap and dispels 0.5 ml by pushing the plunger to the 0.5 ml mark (the first dose). The 2^{nd} nd dose will empty the syringe. One syringe, therefore, will last one day. The patient should apply the medication by starting at the point where the pain is most intense or where noticeable disfigurement about the heel area is located.

The patient's progress should be evaluated every 2 weeks to assess progress, side effects, etc. Although some patients respond to the medication during the first month of therapy, others have responded after 2-3 months of therapy. It is important to not miss doses of medication.

Application to the entire effected area is important. During the treatment regimen, each patient's progress should be evaluated, at least every two weeks. If no results have occurred by the end of the 3rd week, the dose should be increased and/or the medicament applied more often than twice daily.

Since calcium channel blockers may be antihypertensive, the patient's blood pressure should be monitored at the physician's office after the first dose of a calcium channel blocker medicament is applied. To date, however, no changes in blood pressure have been noted.

C. Likely Mechanism of Action and Examples of Efficacy.

It should be noted that Verapamil, a calcium channel blocker, is commonly given orally or intravenously to treat cardiac arrhythmias and/or hypertension. Verapamil is even one of the substances which has been injected directly into the plaque of Peyronie's disease sufferers and applied topically to treat the same. However, despite the other uses for particular fibrotic disorders, it has not heretofore occurred to anyone to compound a topical Verapamil preparation for use in treating Plantar Fasciitis or other maladies which exhibit similar symptoms.

The mechanism of action of the topical calcium channel blocker-based medicaments and methods of treatment are, as yet, unclear to the present inventor and to the clinicians who have thus far used and evaluated the same. The present inventor believes, however, that, upon absorption of the drug through the skin into the collagen degeneration area of the plantar fascia, the calcium channel blocking properties of Verapamil (and other calcium channel blocking agents) causes the body to produce collagenase.

The present belief of the inventor is that the collagen that comprises the majority of the fibrotic tissue disorders contain peptides I, II and III substrates of fibroblast collagenase. When applied transdermally, a calcium channel blocker is absorbed uniformly into the fibrotic tissue (plaque) and prevents divalent calcium ions from entering the cell membranes. The calcium ions that are blocked, as well as zinc ions, bind with the peptides I, II and III substrates of fibroblast collagenase, thereby forming a ternary complex which triggers the completion of the formation of the collagenase that is highly specific for the subject collagen. This collagenase is produced in sufficient quantities to catabolize the collagen. The collagen can be type I, II or III, and the collagenase produced would specifically match each respective type.

Whatever the mechanism, the present medicament shows an astonishing efficacy. One patient involved in experimental evaluation, by way of example, has experienced results not achievable with other regimens. After using the medicament of the present invention, in the prescribed manner, this patient's Plantar Fasciitis symptoms were completely reversed in two week's time. Other experimental patients, albeit exhibiting symptoms to various degrees, have shown equally remarkable and complete recoveries, including one patient who suffered from the disease for several years, never having previously experienced success with any prior treatment regimen undertaken by numerous physicians.

While the initial dose of the preferred Verapamil gel has, to date, been 0.50 ml (containing 40 mg of Verapamil) applied twice daily, in the morning and at night, it is suspected that, once a patient receives relief, the condition may reoccur if the medication is stopped. In that event, continued use of the present medicament, perhaps at a lower dose, or less frequently, may be indicated.

D. Plantar Fasciitis

The use of the present medicaments in the treatment of collagen degeneration associated with repetitive microtears of the plantar fascia is, according to experience to date, identical to the use in treating Plantar Fasciitis. Variations of dosage and periodicity of treatments may be indicated by further research, but the present experiments using identical treatment regimens to those described above for Plantar Fasciitis have yielded a very high rate of positive results.

Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

I claim:

1. A medicament for use in the treatment of tissue disorders involving collagen degeneration, comprising:
a carrier compound for facilitation non-invasive, transdermal delivery of calcium channel blocker agents in human recipients; and
a calcium channel blocker agent dispersed in said carrier means.

2. The medicament of claim 1 wherein said calcium channel blocker agent is at least one calcium channel blocker substance selected from the classes of calcium channel blockers consisting of diphenylalkylamines, benzothiazepines, and dihydropyridines.

3. A method for treating tissue disorders involving collagen degeneration comprising the steps of:
selecting a medicament comprising:
a carrier compound for facilitation non-invasive, transdermal delivery of calcium channel blocker agents in human recipients; and
a calcium channel blocker agent dispersed in said carrier means; and
topically applying said medicament for non-invasive transdermal delivery of said calcium channel blocker agent to a collagen degeneration tissue disorder.

4. The method of claim 3 wherein said calcium channel blocker agent is at least one calcium channel blocker substance selected from the classes of calcium channel blockers consisting of diphenylalkylamines, benzothiazepines, and dihydropyridines.

5. A topical medicament for use in the treatment of heel pain, comprising:
a carrier compound for facilitation non-invasive, transdermal delivery of calcium channel blocker agents in human recipients; and
a calcium channel blocker agent dispersed in said carrier means.

6. The medicament of claim 5 wherein said calcium channel blocker agent is at least one calcium channel blocker substance selected from the classes of calcium channel blockers consisting of diphenylalkylamines, benzothiazepines, and dihydropyridines.
